(19) United States
(12) Patent Application Publication
(10) Pub. No.: US 2021/0024448 A1
(43) Pub. Date: Jan. 28, 2021

(54) PROCESS FOR ESTERIFICATION AND TRANS-ESTERIFICATION REACTIONS

(71) Applicant: BASF SE, Ludwigshafen am Rhein (DE)

(72) Inventors: Michael KOCH, Ludwigshafen (DE); Oliver REICH, Ludwigshafen (DE); Michael SCHIER, Ludwigshafen (DE)

(73) Assignee: BASF SE, Ludwigshafen am Rhein (DE)

(21) Appl. No.: 16/980,267

(22) PCT Filed: Mar. 6, 2019
(86) PCT No.: PCT/EP2019/055596

§ 371 Date: Sep. 11, 2020

(30) Foreign Application Priority Data
Mar. 13, 2018 (EP) 18161389.4

(51) Int. Cl. C07C 67/08; B01J 31/02; B01J 35/12

(52) U.S. Cl. CPC C07C 67/08; B01J 31/0225; B01J 2531/002; B01J 2231/49; B01J 35/12

(57) ABSTRACT

A process for esterification and/or trans-esterification, uses an acid as catalyst in the presence of an anionic surfactant. The process may involve esterifying and/or trans-esterifying at least one fatty acid and/or fatty acid ester with at least one alcohol using at least one acid catalyst, such as methanesulfonic acid, in the presence of at least one anionic surfactant

This invention deals with a process for esterification and/or trans-esterification, using an acid as catalyst in the presence of an anionic surfactant.

Examples of inorganic acid catalysts are acids like sulfuric acid, phosphoric acid, nitric acid, hydrochloric acid, hydrofluoric acid; examples of organic acid catalysts are acids like citric acid, p-toluene sulfonic acid, sulfamic acid, formic acid, acidic acid, propionic acid or alkane sulfonic acids like ethanesulfonic acid or, preferably, methanesulfonic acid (MSA).

Acids, for example alkanesulfonic acids, like methanesulfonic acid (MSA), can be used to catalyze esterification reactions. In the biodiesel industry this is done to convert free fatty acid feedstocks into fatty acid methyl esters (FAME) or fatty acid ethyl esters.

The term “biodiesel” refers in general to a vegetable oil- or animal fat-based diesel fuel, containing mainly long-chain alkyl (e. g. methyl) esters. Usually, biodiesel is made by chemically reacting lipids (triglycerides) (e. g. vegetable oil or animal fat) or fatty acids with an alcohol, resulting in fatty acid esters (e. g. fatty acid methyl esters).

In general, a fatty acid ester (FAE) is an ester of a fatty acid with an alcohol, for example glycerine.

While free fatty acids are converted to biodiesel by acidic catalysis only, the conversion of triglycerides (vegetable oils or animal fats) can be done by either acidic or alkaline catalysis. Historically triglyceride conversion is done with alkaline catalysts, but acidic catalysis, for example with MSA, is also possible under relatively high temperature and pressure. This process is thus in principle known in the art and is described in some publications.

For example, WO 2011/018228 A1 discloses a process for manufacturing biodiesel by acid transesterification, and the use of sulfonic acid, for example methanesulfonic acid, as a catalyst in this process.

Depending on the feedstock, the reaction mixtures for the manufacture of biodiesel may contain MSA, methanol, water, glycerin, fatty acids, triglycerides and FAME. These form a two phase system, one oil phase consisting of fatty acids/oil/FAME, and one aqueous phase consisting of water/methanol/glycerin and MSA.

Since the acid catalyst, for example MSA, is mainly soluble in the aqueous phase, and only sparingly soluble in the oil phase, most of the protons needed for the catalytic action reside in the aqueous phase.

For reactions taking place in a two-phase system (oil/water), the concept of two-phase catalysis (sometimes also called phase-transfer catalysis) is generally known.

For example, EP 1 526 126 A1 describes in general the use of a phase-transfer catalyst system, in a solvent-free process for the manufacture of conjugated, multiply unsaturated fatty acid esters, useful in food additives.

However, since in biodiesel synthesis a proton transfer has to be effected, it was questionable whether this teaching could also be useful in biodiesel synthesis.

US 2004/167343 A1 describes the trans-esterification of triglycerides and polyols, using a phase-transfer catalyst, for example quaternary ammonium salt, and a basic initiator. However, this process comprising a basic initiator is not applicable for acid catalysis.

US 2015/119594 A1 discloses a method for the preparation of fatty acid mono- and di-esters of poly-alcohols, in particular erythritol, by subjecting a fatty acid and erythritol to an esterification reaction in the presence of an acid catalyst, a water carrier and optionally a phase-transfer catalyst, for example a polyether and/or quaternary ammonium salt. However, experiments performed by the inventors of the present invention have shown that this teaching does not work well.

EP 1 870 446 A1 describes a process for the trans-esterification of triglycerides, using a basic activator, like Na OH or KOH, and a phase transfer catalyst selected from certain benzyl-trialkyl ammonium salts and trisalkylmethyl ammonium salts. Since this is a alkaline catalyzed process, it can't be applied to acid catalyzed process as described in the present invention.

As mentioned before, the above processes are associated with certain problems and disadvantages. Inter alia, the solubility of the catalyst in the oil phase remains to be improved.

The object of the present invention therefore was to overcome or reduce, at least partially, the problems and disadvantages mentioned above. In particular, it was an object of the present invention to provide an efficient process for acid catalyzed esterification and/or transesterification of fatty acids and/or fatty acid esters. It was also an object of the present invention to provide a method to increase the catalytic proton concentration in the oil phase during esterification and/or transesterification reactions.

It has now been found that the catalytic activity of acids, for example alkanesulfonic acids, like MSA, in esterification and transesterification reactions can be enhanced by addition of anionic surfactants.

These surfactants can be protonated by MSA, thereby forming a strong acid themselves but having better solubility in the oil phase. Such surfactants allow for a higher protons content in the oil phase and thus enhance catalytic activity.

The problem set out above is solved by the features of the independent claims. Preferred embodiments of the present invention are provided by the dependent claims

In a first aspect the invention relates to a process for the esterification and/or trans-esterification of at least one fatty acid FA and/or fatty acid ester FAE with at least one alcohol, wherein at least one acid A is used as catalyst in the presence of at least one anionic surfactant S, wherein the acid A contains an alkanesulfonic acid, and wherein the alkanesulfonic acid A is methanesulfonic acid.

The present invention also relates to (i) a catalyst for esterification and/or trans-esterification reactions, comprising, preferably consisting of, at least one alkanesulfonic acid A as defined below and at least one anionic surfactant S as defined below, and (ii) the use of at least one alkanesulfonic acid A, as defined below, and at least one anionic surfactant S, as defined below, for the catalysis of esterification and/or trans-esterification reactions, and (iii) the use of at least one anionic surfactant S, as defined below, for the enhancement of catalytic activity of alkanesulfonic acids in esterification and/or trans-esterification reactions.

The present invention also relates to an esterification product, obtainable by the inventive process, and a transesterification product, obtainable by the inventive process.

Anionic surfactant in general means a surfactant with a negatively charged ionic group. Anionic surfactants, in the context of this invention, include, but are not limited to, surface-active compounds that contain a hydrophobic group and at least one water-solubilizing anionic group, usually selected from sulfates, sulfonates, and carboxylates to form a water-soluble compound. Anionic surfactants may be compounds of general formula (VIII), which might be called (fatty) alcohol/alkyl (ethoxy/ether) sulfates [(F)A(E)S] when A- is SO3-, (fatty) alcohol/alkyl(ethoxy/ether) carboxylat [(F)A(E)C] when A- is —RCOO—:

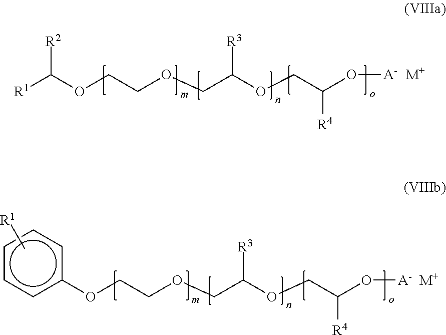

The variables in general formula (VIII) are defined as follows:

R1 is selected from C1-C23-alkyl (such as 1-, 2-, 3-, 4-C1-C23-alkyl) and C2-C23-alkenyl, wherein alkyl and/or alkenyl are linear or branched, and wherein 2-, 3-, or 4-alkyl; examples are n-C7H15, n-C9H19, n-4-C9H19, n-C11H23, n-C13H27, n-C15H31, n-C17H35, i-C9H19, i-C12H25.

R2 is selected from H, C1-C20-alkyl and C2-C20-alkenyl, wherein alkyl and/or alkenyl are linear or branched.

R3 and R4, each independently selected from C1-C16-alkyl, wherein alkyl is linear or branched; examples are methyl, ethyl, n-propyl, isopropyl, n-butyl, isobutyl, sec-butyl, tert-butyl, n-pentyl, isopentyl, sec-pentyl, neopentyl, 1,2-dimethylpropyl, isoamyl, n-hexyl, isohexyl, sec-hexyl, nheptyl, n-octyl, 2-ethylhexyl, n-nonyl, n-decyl, isodecyl.

A- is selected from —RCOO—, —SO3- and —RSO3-, wherein R is selected from C1-C18-alkyl, wherein alkyl is linear or branched.

M+ is selected from salt forming cations. Salt forming cations may be monovalent or multivalent; hence M+ equals 1/v Mv+. Examples include but are not limited to sodium, potassium, magnesium, calcium, ammonium, and the ammonium salt of mono-, di, and triethanolamine (TEA).

The integers of the general formulae (VIIIa) and (VIIIb) are defined as follows:

m is in the range from zero to 200, preferably 1-80, more preferably 3-20; n and o, each independently in the range from zero to 100; n preferably is in the range from 1 to 10, more preferably 1 to 6; o preferably is in the range from 1 to 50, more preferably 4 to 25. The sum of m, n and o is at least one, preferably the sum of m, n and o is in the range from 5 to 100, more preferably in the range of from 9 to 50.

Anionic surfactants of the general formula (VIII) may be of any structure, block copolymers or random copolymers.

Further non-limiting examples of suitable anionic surfactants include salts (M+) of sulfates, sulfonates or carboxylates derived from natural fatty acids such as tallow, coconut oil, palm kernel oil, laurel oil, olive oil, or canola oil. Such anionic surfactants comprise sulfates, sulfonates or carboxylates of lauric acid and/or myristic acid and/or palmitic acid and/or stearic acid and/or oleic acid and/or linoleic acid in different amounts, depending on the natural fatty acids from which the soaps are derived.

Further suitable anionic surfactants include salts (M+) of C12-C18 alkylsulfonic acids, C12-C18 sulfonated fatty acid alkyl esters (such as C12-C18 sulfo fatty acid methyl esters), C10-C18-alkylarylsulfonic acids (such as n-C10-C18-alkylbenzene sulfonic acids) and C10-C18 alkyl alkoxy carboxylates.

M+ in all cases is selected from salt forming cations. Salt forming cations may be monovalent or multivalent; hence M+ equals 1/v Mv+. Examples include but are not limited to sodium, potassium, magnesium, calcium, ammonium, and the ammonium salt of mono-, di, and triethanolamine (TEA).

Non-limiting examples of further suitable anionic surfactants include branched alkylbenzenesulfonates (BABS), phenylalkanesulfonates, alpha-olefinsulfonates (AOS), olefin sulfonates, alkene sulfonates, alkane-2,3-diylbis(sulfates), hydroxyalkanesulfonates and disulfonates, secondary alkanesulfonates (SAS), paraffin sulfonates (PS), sulfonated fatty acid glycerol esters, alkyl- or alkenylsuccinic acid, fatty acid derivatives of amino acids, diesters and monoesters of sulfo-succinic acid.

Anionic surfactants may be compounds of general formula (IX), which might be called N-acyl amino acid surfactants:

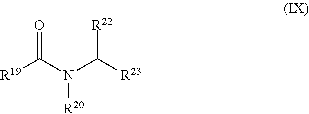

The variables in general formula (IX) are defined as follows:

R19 is selected from C6-C22-alkyl, wherein alkyl is linear or branched.
R20 is selected from H and C1-C4-alkyl.
R21 is selected from methyl, —(CH2)3NHC(NH)NH2, —CH2C(O)NH2, —CH2C(O)OH, —(CH2)2C(O)NH2, —
(CH2)2C(O)OH, (imidazole-4-yl)-methyl, —CH(CH3)C2H5, —CH2CH(CH3)2, —(CH2)4NH2, benzyl, hydroxymethyl, —CH(OH)CH3, (indole-3-yl)-methyl, (4-hydroxy-phenyl)-methyl, and isopropyl.
R22 is selected from —COOX and —CH2SO3X, wherein X is selected from Li+, Na+ and K+.

Non-limiting examples of further suitable N-acyl amino acid surfactants are the mono- and dicarboxylate salts (e.g., sodium, potassium, ammonium and TEA) of N-acylated glutamic acid, for example, sodium cocoyl glutamate, sodium lauroyl glutamate, sodium myristoyl glutamate, sodium palmitoyl glutamate, sodium stearoyl glutamate, disodium cocoyl glutamate, disodium stearoyl glutamate, potassium cocoyl glutamate, potassium lauroyl glutamate, and potassium myristoyl glutamate; the carboxylate salts (e.g., sodium, potassium, ammonium and TEA) of Nacylated alanine, for example, sodium cocoyl alaninate, and TEA lauroyl alaninate; the carboxylate salts (e.g., sodium, potassium, ammonium and TEA) of N-acylated glycine, for example, sodium cocoyl glycinate, and potassium cocoyl glycinate; the carboxylate salts (e.g., sodium, potassium, ammonium and TEA) of N-acylated sarcosine, for example, sodium lauroyl sarcosinate, sodium cocoyl sarcosinate, sodium myristoyl sarcosinate, sodium oleoyl sarcosinate, and ammonium lauroyl sarcosinate.

Anionic surfactants may further be selected from the group of soaps. Suitable are salts (M+) of saturated and unsaturated C12-C18 fatty acids, such as lauric acid, myristic acid, palmitic acid, stearic acid, behenic acid, oleic acid, (hydrated) erucic acid. M+ is selected from salt forming cations. Salt forming cations may be monovalent or multivalent; hence M+ equals 1/v Mv+. Examples include but are not limited to sodium, potassium, magnesium, calcium, ammonium, and the ammonium salt of mono-, di, and triethanolamine (TEA).

Further non-limiting examples of suitable soaps include soap mixtures derived from natural fatty oils such as tallow, coconut oil, palm kernel oil, laurel oil, olive oil, or canola oil. Such soap mixtures comprise soaps of lauric acid and/or myristic acid and/or palmitic acid and/or stearic acid and/or oleic acid and/or linoleic acid in different amounts, depending on the natural fatty acids from which the soaps are derived.

Mixtures of two or more different anionic surfactants may also be present according to the present invention.

Fatty acids suitable for acidic catalyzed esterification according to the present invention are for example C12-C20 saturated or unsaturated fatty acids, preferably, but not limited to, palm fatty acid, oleic acid, linoleic acid, stearic acid, laurylic acid, cetyl acid, or mixtures thereof, such as present in vegetable oils like palm oil, coco nut oil, soybean oil, olive oil, castor oil, sun flower oil, sun flower kernel oil, rape seed oil, including the free fatty acids released from such oils during processing, e.g. saponification, hydrolyzation, or during use as cooking oil (“used cooking oil”) or during processing of animal fats (tallow).

Fatty acid esters suitable for acidic catalyzed trans-esterification according to present invention include vegetable oil triglycerides like palm oil, rape seed oil, soybean oil, coco nut oil, or animal fats (tallow), or mixtures thereof.

In an embodiment of the present invention, the alcohol is selected from the group consisting of mono-alcohols.

In a further embodiment of the present invention, the alcohol is selected from the group consisting of methanol, ethanol, propanol, butanol, and mixtures thereof.

In a preferred embodiment of the invention, the alcohol is methanol and/or ethanol. Preferably, the alcohol contains methanol or consists of methanol.

In an embodiment of the inventive process, the acid A is not a fatty acid.

Preferably, the acid A according to the present invention is selected from the group consisting of sulfuric acid, sulfonic acids and mixtures thereof.

In another embodiment of the process according to this invention, the acid A is selected from the group consisting of sulfonic acids and/or contains an alkanesulfonic acid.

In a preferred embodiment of the inventive process, the acid A is selected from the group consisting of alkanesulfonic acids.

Preferably, the alkanesulfonic acid A contains methanesulfonic acid, more preferably is methanesulfonic acid.

In another preferred embodiment of the inventive process, the alkanesulfonic acid A is methanesulfonic acid, dissolved in water. The alkanesulfonic acid content in water may be more than 60% by weight, preferably more than 70% by weight, more preferably more than 80% by weight, even more preferably more than 90% by weight.

### EXAMPLES

In the following, some examples are described in detail, which may serve to illustrate some aspects of the present invention.

Unless otherwise noted, percentages are given as % by weight.

A) Esterification

Test Series 1

For all tests, a model feedstock (fatty acid triglyceride mixture) of following composition was used: 80% oleic acid, 20% rape seed oil.

Approximately 300 grams of the model feedstock were filled into a 500 ml glass reactor with reflux condenser and dosing unit. The desired amount of methanol was added. Condenser cooling, stirrer and heating were turned on. Once reaction temperature is reached (reflux), the desired amount of catalyst (Lutropur® MSA-XP) and surfactant additive according to present invention was added to start the reaction. After reaction was started, each 30 minutes a sample was taken, the biodiesel and water phase separated in a separating funnel and the biodiesel phases kept for later analysis. After 3 hours the reaction mixture was cooled down and transferred into a separating funnel, where the biodiesel phase and the water phase were separated and a sample of the biodiesel phase taken for analysis.

The esterification was done under the following conditions:

Methanol content: 20% w/w
Lutropur® MSA-XP: 1% (w/w) active matter

### Surfactant: q.s.

Temperature: reflux (approx. 65° C.)
Duration: 3 hours
(Lutropur® MSA-XP is a product of BASF SE, methanesulfonic acid, approximately 94% by weight in water, CAS no. 75-75-2)

All samples from the biodiesel phase taken during the experiment were analyzed for the acid value by titration with KOH according to EN 14104. The lower the acid value, the more free fatty acid have been converted to biodiesel (fatty acid methyl ester; FAME).

The following surfactants (commercially available products) were used.

Dehyquart® SP (Tallow alkyl amine, ethoxylated, phosphates)
Disponil® OCS 27 (Aqueous solution based on: Sulfuric acid, mono(C16-18 and C18-unsatd. alkyl) esters, sodium salts)
Pluriol® E400 (polyethylene glycol, average molar mass 400)
Disponil® OSS 50 KS (9(or 10)-Sulphooctadecanoic acid, potassium salt)
Lutensit® A-EP (Oxirane, methyl-, polymer with oxirane, mono-C10-16-alkyl ethers, phosphates)
Aliquat® 336 (Quaternary ammonium compounds, tri-C8-10-alkylmethyl, chlorides)
Disponil® SUS IC 10 (Na salt of Di-isodecylsulphosuccinate)
Aliquat® 175 (Tributylmethylammonium chloride)
Disponil® LDBS 55 (Benzenesulfonic acid, C10-13-alkyl derivs., sodium salts)

The experimental results (acid values) are shown in table 1.

TABLE 1

| | Acid Value |
|---|---|
| Surfactant | [mg KOH/g] |
| No surfactant (1% MSA only) | 48.9 |
| Pluriol E400 | 45.9 |
| Aliquat 336 | 46.0 |
| Aliquat 175 | 47.2 |
| Lutensit A-EP | 47.9 |
| Disponil OSS 50 KS | 28.4 |
| Disponil SUS IC 10 | 16.5 |
| Disponil LDBS 55 | 18.0 |

The best results were achieved with Disponil® OSS 50 KS, Disponil® SUS IC 10 and Disponil® LDBS 55, which could increase the catalytic activity of MSA significantly.

These three surfactants stem from the class of sulfonate-based surfactants.

Test Series 2

Using the same methodology as in test series one, the surfactant concentrations were varied at a constant MSA level of 1% w/w (active). Only the three best surfactants from test series 1 were used.

The results of this test series 3 are shown in table 2.

TABLE 2

| | Acid Value |
|---|---|
| Surfactant | [mg KOH/g] |
| No surfactant | 48.9 |
| (1% MSA only) | |
| 0.2% Disponil OSS 50 KS | 38.7 |
| 0.5% Disponil OSS 50 KS | 37.7 |
| 0.2% Disponil SUS IC 10 | 35.0 |
| 0.5% Disponil SUS IC 10 | 21.8 |
| 0.2% Disponil LDBS 55 | 33.3 |
| 0.5% Disponil LDBS 55 | 22.0 |

All processes using a surfactant additive additionally to MSA showed better performance than MSA stand-alone, both at 0.2% w/w and 0.5% w/w of the additive.

Test Series 3

Tests were carried out using the same methodology as in test series 1. In this test series 4, MSA concentrations and surfactant concentrations were varied and compared to MSA stand-alone. Disponil® SUS IC 10 was used for these tests.

This resulted in following catalyst combinations:

| | | Disponil ® |
|---|---|---|
| | MSA | SUS IC 10 |
| Cat 0.7/0.2 | 0.7% w/w | 0.2% w/w |
| Cat 0.7/0.5 | 0.7% w/w | 0.5% w/w |
| Cat 0.5/0.2 | 0.5% w/w | 0.2% w/w |
| Cat 0.5/0.5 | 0.5% w/w | 0.5% w/w |
| 0.5% MSA | 0.5% w/w | 0.0% w/w |
| 0.7% MSA | 0.7% w/w | 0.0% w/w |
| 1% MSA | 1.0% w/w | 0.0% w/w |

“Cat 0.5/0.5” performed better than 1% MSA, despite the overall proton concentration being less in this catalyst combination.

The results are shown in table 3:

TABLE 3

| | Acid Value |
|---|---|
| Cat System | [mg KOH/g] |
| Cat 0.7/0.2 | 40 |
| Cat 0.7/0.5 | 29 |
| Cat 0.5/0.2 | 49 |
| Cat 0.5/0.5 | 34 |
| 0.5% MSA | 58 |
| 0.7% MSA | 52.9 |
| 1% MSA | 41.8 |

Test Series 4 (Sulfur Content)

Various samples of FAME from above test series were washed with water until washing water was neutral, and then analyzed for sulfur content.

If the surfactants would not wash out as easily as MSA, the resulting biodiesel would be contaminated with surfactants, increasing the sulfur content and potentially impacting phase separation.

| | sulfur |
|---|---|
| | content of |
| | FAME |
| Catalyst system | (mg/Kg) |
| MSA (1%) + Disponil ® SUS IC 10 (0.2%) | 17 |
| MSA (1%) + Disponil ® SUS IC 10 (0.5%) | 20 |
| MSA (0.7%) + Disponil ® SUS IC 10 (0.2%) | 29 |
| MSA (0.7%) + Disponil ® SUS IC 10 (0.5%) | 17 |
| MSA (0.5%) + Disponil ® SUS IC 10 (0.2%) | 18 |
| MSA (0.5%) + Disponil ® SUS IC 10 (0.5%) | 22 |
| MSA (0.5%) | 24 |
| MSA (0.7%) | 12 |
| MSA (1%) | 13 |

The sulfur content of all tested samples was similar. There is no indication of increased sulfur content when combining MSA with a sulfonic acid based surfactant, thus there are no unwanted surfactants in the biodiesel phase after reaction and washing.

B) Trans-Esterification

It was tested if the positive effects found for esterification would also be found under transesterification conditions. The following process was utilized:

All ingredients were weight into an autoclave. The autoclave was closed, stirrer and heating started and the reaction run for 3 hours. Afterwards, the reaction mixture was transferred into a separating funnel and biodiesel phase and water/glycerin phase separated. The acid value of the biodiesel phase was then determined as described above. The lower the acid value, the better conversion to FAME. Additionally, samples were analyzed to determine the actual FAME content.

The transesterification reactions were carried out under the following conditions:

Model feedstock
50% oleic acid
50% rape seed oil

### Methanol: 30% w/w

Lutropur® MSA-XP: 1% w/w active matter
Surfactant: 1% w/w active matter

### Temperature: 130° C.

Pressure: approx. 3.5 bar
Duration: 3 hours

Both tested additives, Disponil OSS 50 KS and Disponil LDBS 55, exhibited in combination with MSA lower acid values and higher FAME content compared to MSA stand-alone. Thus these surfactants increased the overall yield of the transesterification, despite the amount of catalytically active protons being the same in all three experiments. The results are shown in table 4:

TABLE 4

| | Acid Value | FAME |
|---|---|---|
| | [mg KOH/g] | [%] |
| MSA | 7.1 | 81.9 |
| Disponil OSS 50 KS | 6.2 | 89.6 |
| Disponil SUS IC 10 | 8.2 | 84.6 |
| Disponil LDBS 55 | 5.0 | 90.5 |

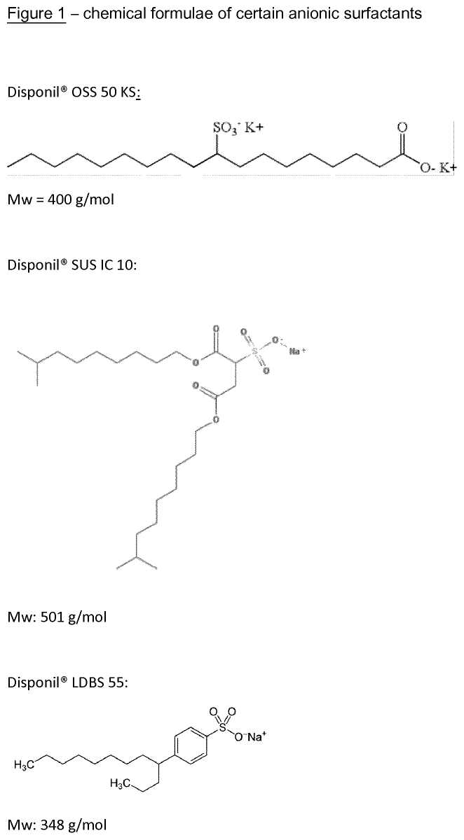

FIG. 1 shows, for illustration purposes, the chemical formulae of some exemplary anionic surfactants.

1. A process, comprising:
esterifying and/or trans-esterifying a first reagent comprising a fatty acid and/or a fatty acid ester with a second reagent comprising an alcohol using a catalyst comprising methanesulfonic acid in the presence of an agent comprising an anionic surfactant.

2. The process of claim 1, wherein the alcohol comprises a mono-alcohol.

3. The process of claim 1, wherein the alcohol comprises methanol, ethanol, propanol, and/or butanol.

4. The process of claim 1, wherein the alcohol comprises methanol.

5. The process of claim 1, wherein the alcohol is methanol.

6. The process of claim 1, wherein the fatty acid is different from the methanesulfonic acid.

7. The process of claim 1, wherein the fatty acid comprises a saturated fatty acid and/or an unsaturated fatty acid.

8. The process of claim 1, wherein the fatty acid comprises 12 to 20 carbon atoms.

9. The process of claim 1, wherein the fatty acid comprises a saturated fatty acid and/or an unsaturated fatty acid comprising 12 to 20 carbon atoms.

10. The process of claim 1, wherein the fatty acid comprises oleic acid, palm fatty acid, linoleic acid, stearic acid, laurylic acid, and/or cetyl acid.

11. The process of claim 1, wherein the fatty acid is obtained from rape seed oil, soy bean oil, tallow, used cooking oil, and/or palm distilled fatty acid.

12. The process of claim 1, wherein the fatty acid ester comprises a saturated fatty acid ester and/or an unsaturated fatty acid ester.

13. The process of claim 1, wherein the fatty acid ester comprises 12 to 20 carbon atoms.

14. The process of claim 1, wherein the fatty acid ester comprises an ester of a saturated fatty acid comprising 12 to 20 carbon atoms and/or an unsaturated fatty acid comprising 12 to 20 carbon atoms.

15. The process of claim 1, wherein the fatty acid ester comprises an ester of glycerine.

16. The process of claim 1, wherein the fatty acid ester comprises a triglyceride.

17. The process of claim 1, wherein the fatty acid ester comprises rape seed oil, soy bean oil, palm oil, palm kernel oil, coconut oil, sunflower oil, sunflower kernel oil, castor oil, olive oil, canola oil, and/or tallow.

18-20. (canceled)

21. The process of claim 1, wherein the methanesulfonic acid is dissolved in water.

22. The process of claim 1, wherein the methanesulfonic acid is dissolved in water, and
wherein the methanesulfonic acid has a concentration in the water of more than 60 wt %.

23. The process of claim 1, wherein the anionic surfactant comprises a hydrophobic group and a water-solubilizing anionic group comprising a sulfate, sulfonate, and/or carboxylate.

24. The process of claim 1, wherein the anionic surfactant has a molecular weight Mw in a range of from 300 to 600 g/mol, and/or
wherein the anionic surfactant comprises a sulfonate.

25. (canceled)

26. The process of claim 1, wherein the anionic surfactant comprises a salt of a C12-C18 alkylsulfonic acid, a salt of a C12-C18 sulfonated fatty acid alkyl ester, a salt of a C10-C18-alkylarylsulfonic acid, and/or a salt of a C10-C18 alkyl alkoxy carboxylate.

27. The process of claim 1, wherein the methanesulfonic acid, is in an amount in a range of from 0.1 to 5 wt %, relative to an oil phase weight.

28. The process of claim 1, wherein the anionic surfactant is in an amount in a range of from 0.05 to 3 wt. %, relative to an oil phase weight.

29. The process of claim 1, wherein the esterifying and/or trans-esterifying is done in an open or closed system, at a temperature in a range of from 60 to 150° C.

30. The process of claim 1, wherein the alcohol is added in a stochiometric molar excess in a range of from 1 to 20 times and/or is added continuously until reaction is finished.

31. The process of claim 1, wherein the reaction is done in batches with subsequent separation of oil and a water/glycerin/alcohol phase, or continuously with constant evaporation of a water/alcohol.

32. A catalysts configured for a esterification and/or trans-esterification reaction, comprising;
methanesulfonic acid A; and
an anionic surfactant comprising a hydrophobic group and a water-solubilizing anionic group,
wherein the water-solubilizing anionic group comprises a sulfate, sulfonate, and/or carboxylate.

33. (canceled)

34. A process for enhancing a catalytic activity of the methanesulfonic acids in the esterifying and/or trans-esterifying of the process of claim 1, the method comprising:
combining with the methanesulfonic acid the anionic surfactant which comprises a hydrophobic group and a water-solubilizing anionic group,
wherein the water-solubilizing anionic group comprises a sulfate, sulfonate, and/or carboxylate.

35-36. (canceled)
